(19) United States
(12) Patent Application Publication
(10) Pub. No.: US 2024/0423595 A1
(43) Pub. Date: Dec. 26, 2024

(54) APPARATUSES FOR REMOVAL OF A VALVE CLIP

(71) Applicant: KONINKLIJKE PHILIPS N.V., EINDHOVEN (NL)

(72) Inventors: Nathan C FRANCIS, COLORADO SPRINGS, CO (US); Michael ANDERSON, COLORADO SPRINGS, CO (US)

(21) Appl. No.: 18/709,740

(22) PCT Filed: Nov. 17, 2022
(86) PCT No.: PCT/EP2022/082171

§ 371 Date: May 13, 2024

Related U.S. Application Data
(60) Provisional application No. 63/282,080, filed on Nov. 22, 2021.

(51) Int. Cl. A61B 17/00; A61B 17/32

(52) U.S. Cl. CPC A61B 17/00234; A61B 17/32; A61B 2017/00243; A61B 2017/00305; A61B 2017/00353; A61B 2017/00358; A61B 2017/00367

(57) ABSTRACT

Systems and Apparatuses for valve clip removal that includes a catheter configured with a first lumen, and a second lumen; a cutting sheath configured with a cutting mechanism; wherein the first lumen is used to deploy a first snare that enables securing a first section of a valve clip; wherein the second lumen is used to deploy a second snare that enables securing of a second section of the valve clip; upon securing the first section of the valve clip, the second snare pulls the cutting sheath over part of the second section of the valve clip for the cutting sheath to: capture part of a second section of valve clip and grasp the valve clip in a stable position, and execute a removal action across the part of the second section of valve clip to detach the valve clip from a valve leaflet whilst the valve clip is grasped; and after performing the removal action and liberating the valve clip from the valve leaflet, the cutting sheath and grasped valve clip are removed through the catheter.

### TECHNICAL FIELD

Embodiments of the subject matter described herein relate generally to medical devices, and more particularly, embodiments of the subject matter relate to systems, apparatuses, and methods for removal of a valve clip used in treating valvular heart disease.

### BACKGROUND

The mitral valve is a two-leaflet valve between two chambers of the heart, the left atrium, and the left ventricle. Mitral valve repair is a treatment for mitral valve disease that includes a leaking (regurgitant) mitral valve and a narrowed (stenotic) mitral valve.

Surgical repair of the mitral valve with the valve clip is an accepted procedure for effective treatment of moderate and severe mitral regurgitation for a certain segment of the population. The valve clip is a transcatheter therapeutic option that is used for the treatment of mitral valve regurgitation, and it entails the edges of two valve leaflets being clipped together which can be performed via a minimally invasive surgical process that results in effective reduction orifice area and therefore reduces regurgitation. A drawback to this surgical intervention is with the implantation of the valve clip there are limited options for transcatheter surgical removal of the clip in the future, which may prevent patients where the valve clip treatment is not successful from receiving more invasive surgical interventions such as a transcatheter mitral valve replacement (TMVR).

It is desirable to present more options for valve clip removal by use of configured surgical instruments and processes that allow for the convenient removal of the mitral valve clip in a minimally invasive surgical setting.

It is also desirable to present options for valve clip removal with configured instruments for the removal of the tricuspid valve clip in a minimally invasive surgical setting.

It is desirable to implement apparatuses for valve clip removal that uses a cutting sheath and two (accessory) tool lumens that enables a combination of snares or graspers to secure the valve clip before cutting or selectively lacerating a portion of the second mitral leaflet that is attached to half of the valve clip.

It is desirable to provide a valve clip removal system that implements a combination of snares and/or graspers for proper aligning/stabilizing an implanted valve clip for performing a surgical laceration for detaching the valve clip, and for capturing the valve clip

It is desirable to implement the system, and apparatus with a cutting sheath portion of the catheter system that can be either laser or mechanical powered. The catheter system will require certain snares depending on the vascular access chosen to reach the clips. The cutting sheath will need a deflectable feature or introducer if a venous/trans-septal approach is used

The above information disclosed in this Background section is only for enhancement of the understanding of the background of the present disclosure and therefore it may contain information that does not form the prior art that is already known in this country to a person of ordinary skill in the art.

### SUMMARY

Disclosed herein are systems and apparatuses of the valve clip removal system.

In accordance with an aspect of the present invention, a valve clip removal system includes a catheter configured with a first lumen, and a second lumen; a cutting sheath configured with a cutting mechanism; wherein the first lumen is used to deploy a first snare that enables securing a first section of a valve clip; wherein the second lumen is used to deploy a second snare that enables securing of a second section of the valve clip; upon securing the first section of the valve clip, the second snare pulls the cutting sheath over part of the second section of the valve clip for the cutting sheath to: capture part of a second section of valve clip and grasp the valve clip in a stable position, and execute a removal action across the part of the second section of valve clip to detach the valve clip from a valve leaflet whilst the valve clip is grasped; and after performing the removal action and liberating the valve clip from the valve leaflet, the cutting sheath and grasped valve clip are removed through the catheter.

The above-mentioned and other features and advantages described herein, and the manner of attaining them will become more apparent and will be better understood by reference to the following description of embodiments of the invention taken in conjunction with the accompanying drawings.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures, which may be illustrated for simplicity and clarity and are not necessarily drawn to scale.

FIG. 1 illustrates a diagram of an exemplary valve clip removal system for removing a valve clip using an apical approach in accordance with various embodiments;

FIG. 2 illustrates a diagram of an exemplary valve clip removal system for removing a valve clip using a venous transseptal approach in accordance with various embodiments;

FIG. 3 illustrates a diagram of an exemplary valve clip removal system removing a valve clip for either the apical or venous transseptal approach in accordance with various embodiments; and

FIG. 4 illustrates a diagram of an exemplary valve clip removal system removing a valve clip with two snare wires within a single snare sheath for the apical approach in accordance with various embodiments.

The exemplifications set out herein illustrate preferred embodiments of the invention, and such exemplifications are not to be construed as limiting the scope of the invention in any manner.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting but are merely representative. The various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Mitral regurgitation (MR) is, by occurrence, the second most common valvular heart disease in the Western world, with a significant impact on prognosis and mortality. A significant number of patients with significant mitral incompetence cannot be submitted to conventional surgery due to high surgical risk.

The surgical implantation of the valve clip is a transcatheter therapeutic option for mitral valve regurgitation, where the edges of the two valve leaflets are clipped together, effectively reducing orifice area and therefore regurgitation. There is a drawback to this therapy as there are few (or likely no available) transcatheter methods for removing the clip in the future, blocking the ability of the patient to receive a transcatheter mitral valve replacement (TMVR). The future surgical intervention therefore can require inserting a second clip or other more invasive type surgical interventions.

The present disclosure describes systems, and apparatuses that allow for a transcatheter method of removing the valve clip (i.e., for the Mitral valve clip or the Tricuspid valve clip) and this can allow surgical options such as transcatheter mitral or tricuspid valve replacement (TMVR/TTVR) for patients previously implanted with valve clip.

The present disclosure describes systems, and apparatuses that involve a cutting sheath with lumen dimensions comparable to a closed valve clip cross-section and two (accessory) tool lumens (or a cutting sheath configured larger enough for 1 or 2 snare catheters to pass down the center), then a combination of snares or graspers to secure the clip prior to cutting. The cutting sheath portion of the catheter system can be configured with either a laser or a mechanically powered device. The catheter system will require certain snares depending on the vascular access chosen to reach the clips. The cutting sheath will also require a deflectable feature or introducer if a venous/trans-septal approach is used for the clip removal procedure.

While the present disclosure describes systems, and apparatuses describes removing the Mitral valve clip, it is contemplated that the present disclosure described systems and apparatuses are not limited to applications for clip removal for only the Mitral valve clip, but are applicable for removal (as similarly described) for the clip removal for the Tricuspid valve with little if any modifications in the surgical instruments (with an exception of the surgical procedure because of the different anatomy).

While the present disclosure describes systems and apparatuses for removing the valve clip using two accessory lumens (for transport of a pair of snares) with a cutting sheath, it is contemplated that alternate embodiments can be configured for transport of the snares that include using a cutting sheath of sufficient diameter (or large enough) to allow for or include two lasso snares to secure the valve clip or to include a single grasping forceps to secure a proximal portion of the valve clip in the transseptal approach.

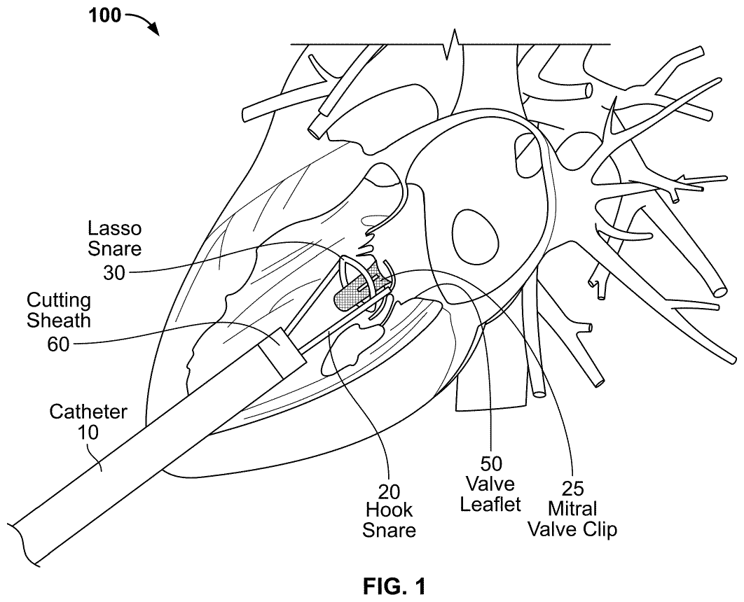

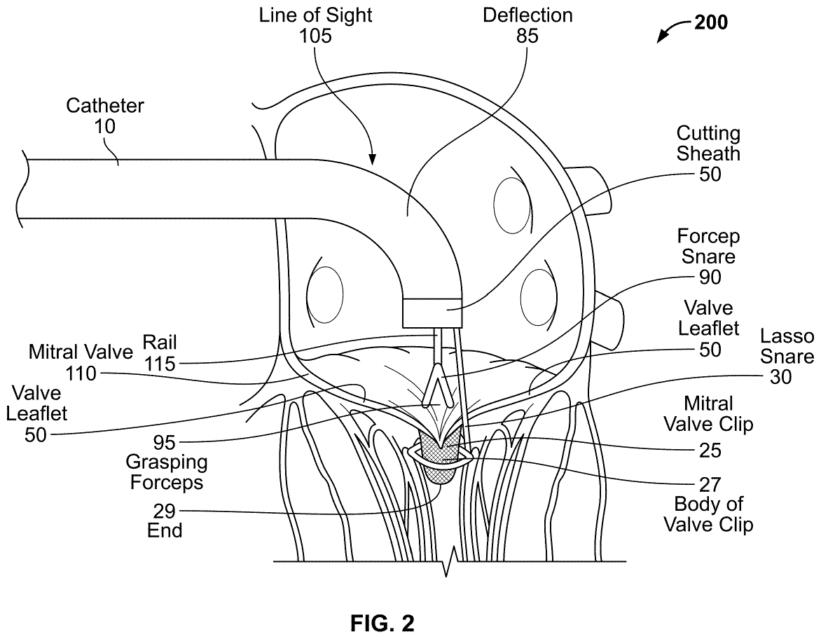

FIG. 1 illustrates a diagram of an exemplary valve clip removal system for removing a valve clip using an apical approach in accordance with various embodiments. In FIG. 1, the valve clip removal system 100 includes a catheter 10, a cutting sheath 60, and two snares: one lasso-type snare (lasso snare) 30 and one hook-type snares (hook snare) 20. In embodiments, the cutting sheath 60 is either a laser or a mechanical device and is configured like a GlideLight or TightRail catheter for endoscopic insertion in the procedure. The snares are a combination of lasso-type, grasping forceps-type (alligator or rat-tooth), and hook-type. There are two approaches a medical provider may use to access the Valve clip for removal. In a transapical or apical approach to access an aortic valve for a valve clip removal procedure, the second snare (if required) is a hook-type snare shown here in FIG. 1. While in the other approach, the venous-transseptal approach, the second type of snare is a forceps-type snare shown in FIG. 2.

In embodiments, for the apical approach, the medical provider will make a small incision between the ribs to access the apex of the heart. At the location where the small incision is made, the medical provider will then thread an introducer for placement into the left ventricle (LV). The Valve clip removal system 100 is then placed through the introducer into the LV as depicted in FIG. 1. For the apical approach depicted in FIG. 1, a lasso-type snare of an approximate diameter in the range of 10-20 mm (for securing the lower part or first section of the clip) and a hook-type snare 20 are required to secure the clip enough to slide the cutting sheath over it. The lasso-type snare 30 secures the lower body of the clip, however, due to the direction of pull on the snare, the clip will torque. Therefore, the second snare, the hook-type snare 20 is required and is threaded up through the mitral valve into the left atrium (LA) and lowered onto the top of the valve clip 25, giving it a straight downward force into the cutting sheath 60. The combination of the two snares will provide the stability required to get a concentric orientation to the cutting sheath. Once the clip is secured, the cutting sheath 60 is progressed forward over the valve clip 25, activating the cutting mechanism to remove the valve clip 25 from the valve leaflet 50. Once the cutting sheath 60 has progressed through the valve leaflet(s) 50 into the LA, the entire system is then removed. All aspects of the valve clip removal procedure can be guided by transesophageal echocardiography (TEE) and fluoroscopy.

FIG. 2 illustrates a diagram of an exemplary valve clip removal system for removing a valve clip using a venous transseptal approach in accordance with various embodiments. The transseptal approach for the Valve clip Removal System (like the aptical approach) consists of a deflectable cutting sheath and two snares, however, a forceps-type snare 90 is used instead of the hook type snare depicted in FIG. 1.

In FIG. 2, in exemplary embodiments, a deflection of the catheter 10 is required for the second approach to retrieve the Valve clip via the venous-transseptal approach and requires the catheter 10 to traverse a pathway similarly and like a method in which the valve clip had originally been implanted. In this approach, vascular access is gained through the right femoral vein into the inferior vena cava (IVC) via an incision; and then a septal puncture needle is threaded to the right atrium (RA) and pierced through the atrial septal wall. The delivery system is then passed through the septal wall into the LA and deflected caudally (or beneath the valve leaflet that was clipped) to give a line of sight 105 to the Valve clip 25 (of FIG. 2). In this exemplary embodiment, the cutting sheath 60 requires deflectability 85 to make the transition from IVC to a septal wall, then septal wall down to mitral valve. This is achieved through modified laser fiber bundling for laser sheath or laser-cut hypotube for a mechanical cutting sheath. The cutting sheath 60 itself could contain the mechanism for deflection or be inserted through a deflectable sheath of adequate size.

In exemplary embodiments, as explained, the pair of snares required (or used) for the transseptal approach is contemplated as a lasso-type snare 30 and a forceps-type snare 90, most likely a rat-tooth shape grasping forceps. The lasso-type snare 30 is extended past the mitral valve 110, between the valve leaflets 50, beyond the end 29 of the valve clip 25, then secured around the body 27 of the valve clip. Again because of the offset direction of pull on the lasso-type snare 30, a second snare (the forceps-type snare 90) is required to secure the clip. The grasping forceps 95 are extended to the top 23 of the valve Clip to secure it and provide a good (or convenient guide) rail 115 to slide the cutting sheath 60 over and position the cutting sheath 60 for the cutting operation to free the valve clip. Once the valve clip 25 is secured by the snares, again the cutting sheath 60 is extended over the clip, activating the cutting mechanism, and freeing the clip from the leaflet tissue. Once valve clip 25 is free or liberated from valve leaflets 50, the entire removal system is pulled out of the heart and out of the body.

In another alternate exemplary embodiment, a pair of snares may be used to secure the clip, or a single grasping forceps can be used to secure a proximal portion of the clip in the transseptal approach.

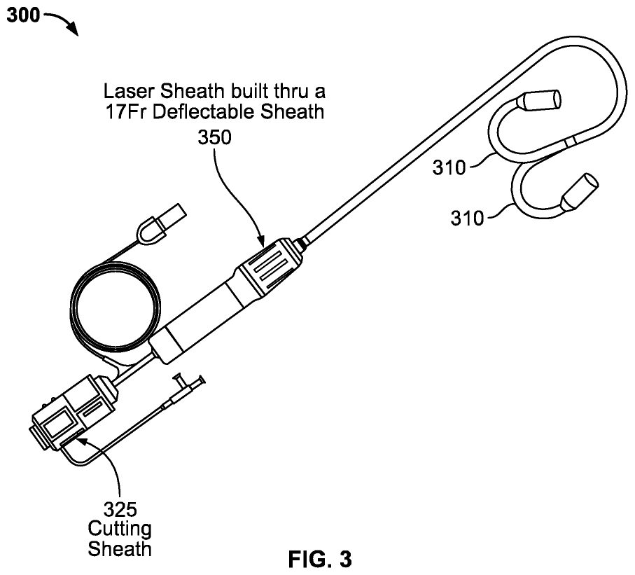

FIG. 3 illustrates a diagram of an exemplary mitral valve clip removal system removing a valve clip for either the apical or venous transseptal approach in accordance with various embodiments. FIG. 3 depicts an exemplary embodiment of a valve clip removal system 300 composed of a pair of accessory tool lumens 310 snares that may include a lasso-type snare, and a second snare that is either a hook-type snare or a forceps-type snare, and a cutting sheath 325 to remove a valve clip. The cutting sheath is shown to include a laser sheath 350 of a modified laser fiber bundling or laser-cut hypotube for the mechanical cutting sheath.

In an alternate exemplary embodiment, the cutting sheath 325 may be configured large enough to pass 1 or 2 snare catheters down the center, rather than using the pair of accessory tool lumens 310.

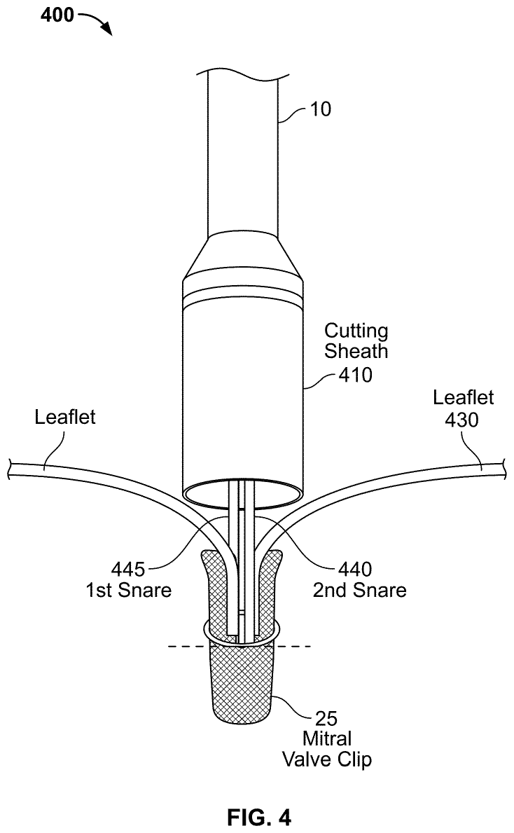

FIG. 4 illustrates a diagram of an exemplary valve clip removal system removing a valve clip with two snare wires within a single snare sheath for the apical approach in accordance with various embodiments. In FIG. 4., in an exemplary embodiment, in the valve clip removal system 400, a catheter 10 is configured with a cutting sheath 410 for deploying a pair of snares 440, 445 to secure and enable the removal of the valve clip 25. In exemplary embodiments, the pair of snares 440, 445 are gooseneck snares. In embodiments, a pair of snare wires can be used as guide/rails for a cutting tip within a cutting sheath 410 for the clip removal and extraction. The system can be integrated into the fluoroscopy suite to display relevant data on a fluoroscopy display device.

In exemplary an embodiment, a valve clip removal system described includes a catheter configured with a first accessory lumen, and a second accessory lumen; a cutting sheath configured with a cutting mechanism; wherein the first accessory lumen is used to deploy a first type of snare that enables securing a first section of a valve clip; wherein the second accessory lumen is used to deploy a second type of snare that enables securing of an second section of the valve clip; the second type of snare is enabled to exert a straight downward force towards the cutting sheath to cause the cutting sheath to progress in direction to a position over part of the second section of the valve clip to enable the cutting sheath to: capture part of an second section of valve clip and grasp the valve clip in a stable position, and execute a removal action across the part of the second section of valve clip to detach the valve clip from a mitral valve leaflet whilst the valve clip is grasped; and after performing the removal action and liberating the valve clip from the mitral valve leaflet, the cutting sheath is (simultaneously) removed with a grasped valve clip through the catheter.

The first type of snare is a lasso-type snare to secure the first section of the valve clip. The second type of snare is a hook-type snare that is hooked to the second section of the valve clip. In a transapical approach to access the mitral valve for a valve clip removal, the lasso-type snare and hook-type snare is used, and the lasso-type snare is configured in a range of 10 mm to 20 mm in diameter. The hook-type snare is configured to attach to the first section of the valve clip and to exert a straight upward force to compensate for an offset direction caused by the torque action to the first section of the valve clip, and to redirect the valve clip for positioning so the cutting sheath can progress forward over the valve clip for a clip removal procedure. The cutting sheath is configured to activate a cutting mechanism to liberate the valve clip from a mitral valve leaflet. A combination of the first and second types of snares provides stability for a concentric orientation for the cutting sheath to progress over the valve clip. The second type of snare is a forceps-type snare that is extended to the top of the second section of the valve clip to secure the valve clip via a grasping action. The forceps-type snare is configured to attach to the first section of the valve clip and to exert the straight upward force to compensate for the offset direction caused by the torque action to the first section of the valve clip, and to redirect the valve clip for positioning so the cutting sheath can progress forward over the valve clip for a valve clip removal procedure. The forceps-type snare is configured to provide a rail that enables the cutting sheath to slide over the forceps-type snare and to progress forward over the valve clip. In a venous-transseptal approach to access a valve for a valve clip removal because of a deflectability required by the cutting sheath for positioning concentrically over a valve clip, the lasso-type snare and the forceps-type snare are used. The cutting sheath is configured as a laser fiber bundle for a laser sheath or a laser-cut hypotube for a mechanical cutting sheath that contains the cutting mechanism for deflection and is insertable through a deflectable sheath of adequate size while achieving the deflectability required by the cutting sheath to access the valve for the valve clip removal.

In another alternate exemplary embodiment, a pair of lasso snares may be used to secure the clip, or a single grasping forceps can be used to secure a proximal portion of the clip in the transseptal approach.

In another exemplary embodiment described, a valve clip removal system includes a catheter configured with a first lumen, and a second lumen; a cutting sheath configured with a cutting mechanism; wherein the first lumen is used to deploy a first snare that enables securing a first section of a valve clip; wherein the second lumen is used to deploy a second snare that enables securing of a second section of the valve clip; upon securing the first section of the valve clip, the second snare pulls the cutting sheath over part of the second section of the valve clip for the cutting sheath to: capture part of a second section of valve clip and grasp the valve clip in a stable position, and execute a removal action across the part of the second section of valve clip to detach the valve clip from a valve leaflet whilst the valve clip is grasped; and after performing the removal action and liberating the valve clip from the valve leaflet, the cutting sheath and grasped valve clip are removed through the catheter.

In another exemplary embodiment described, a valve clip removal apparatus includes a catheter configured with a first accessory lumen and a second accessory lumen; a cutting sheath configured with a cutting mechanism; the first accessory lumen is used to deploy the first type of snare that enables securing a lower part of a valve clip; the second accessory lumen is used to deploy the second type of snare that enables securing of an upper part of the valve clip; the second type of snare is enabled to exert a straight downward force towards the cutting sheath to cause the cutting sheath to progress over the upper part of the valve clip to enable the cutting sheath to hold the valve clip in a stable position and to detach the valve clip from a valve leaflet whilst the valve clip is held; and after the valve clip is freed from the valve leaflet, the cutting sheath is configured for removal whilst the valve clip is held. In response to a torque action caused to valve clip when the lower part is secured by the lasso-type snare, either a hook-type snare or the forceps-type snare is configured to attach to the upper part of the valve clip and to pull or grasp the valve clip for a concentric orientation for a clip removal procedure.

In another exemplary embodiment of a valve clip removal system described, the system includes a pair of accessory tool lumens snares composed of a first snare of a lasso-type snare, and a second snare that is either a hook-type snare or a forceps-type snare, and a cutting sheath to remove a valve clip, the valve clip removal system including a catheter composed of a pair of accessory lumens; the cutting sheath is configured with a cutting mechanism, a first accessory lumen of a pair of accessory lumens deploys the first snare to secure a lower part of a valve clip; a second accessory lumen of the pair of accessory lumens deploys the second snare to secure an upper part of the valve clip; When securing the lower part of the valve clip, the second snare attaches to the upper part of the valve clip to pull the cutting sheath downward over the upper part of the valve clip in order for the cutting sheath to both hold and remove the valve clip from a valve leaflet whilst the valve clip is held; after the valve clip is detached from the valve leaflet, the cutting sheath is configured for removal whilst holding the valve clip. The second snare is not deployed and the valve clip is secured by only the first snare and the cutting sheath for a valve clip removal procedure.

When a torque action is caused to valve clip when the lower part is secured by the lasso-type snare, either the hook-type snare or the forceps-type snare is configured to attach to the upper part of the valve clip and to pull or grasp the valve clip for a concentric orientation for a valve clip removal procedure. In a transapical approach to access the mitral valve for a valve clip removal procedure, the second snare is a hook-type snare; and in a venous-transseptal approach to access the mitral valve for the valve clip removal, the second type of snare is a forceps-type snare.

In another exemplary embodiment, a cutting sheath can be configured with a lumen large enough to pass one or two snare catheters down the center, rather than using two accessory lumens.

For the sake of brevity, conventional techniques related to laser atherectomy, and other functional aspects of the subject matter may not be described in detail herein. Also, certain terminology may be used herein for reference only, and thus is not intended to be limiting. For example, terms such as “first,” “second,” and other such numerical terms referring to structures do not imply a sequence or order unless indicated by the context. The foregoing description may also refer to elements or nodes or features being “connected” or “coupled” together. As used herein, unless expressly stated otherwise, “coupled” means that one element/node/feature is directly or indirectly joined to (or directly or indirectly communicates with) another element/node/feature, and not necessarily mechanically.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. For example, the subject matter described herein is not limited to the infusion devices and related systems described herein. Moreover, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application. Accordingly, details of the exemplary embodiments or other limitations described above should not be read into the claims absent a clear intention to the contrary.

What is claimed is:

1. A valve clip removal system, comprising:
a catheter configured with a first lumen, and a second lumen;
a cutting sheath configured with a cutting mechanism;
wherein the first lumen is used to deploy a first snare that enables securing a first section of a valve clip;
wherein the second lumen is used to deploy a second snare that enables securing of a second section of the valve clip;
upon securing the first section of the valve clip, the second snare pulls the cutting sheath over part of the second section of the valve clip for the cutting sheath to:
capture part of a second section of valve clip and grasp the valve clip in a stable position, and
execute a removal action across the part of the second section of valve clip to detach the valve clip from a valve leaflet whilst the valve clip is grasped; and
after performing the removal action and liberating the valve clip from the valve leaflet, the cutting sheath and grasped valve clip are removed through the catheter.

2. The valve clip removal system of claim 1, further comprising:
wherein the first snare is a lasso-type snare that is configured to secure the first section of the valve clip.

3. The valve clip removal system of claim 2, further comprising:
wherein the second snare is a hook-type snare that is hooked to the second section of the valve clip.

4. The valve clip removal system of claim 3, further comprising:
wherein in a transapical approach to access the mitral valve for a valve clip removal, the lasso-type snare and the hook-type snare are used, and the lasso-type snare is configured in a range of 10 mm to 20 mm in diameter.

5. The valve clip removal system of claim 3, further comprising:
in response to a torque action caused to the valve clip when the first section is secured by the lasso-type snare, the hook-type snare is configured to attach to the first section of the valve clip and to exert a straight upward force to compensate for an offset direction caused by the torque action to the first section of the valve clip, and to redirect the valve clip for positioning so the cutting sheath can progress forward over the valve clip for a clip removal procedure.

6. The valve clip removal system of claim 4, further comprising:
after progression of the cutting sheath to a position over the valve clip, the cutting sheath is configured to activate a cutting mechanism to liberate the valve clip from the valve leaflet coupled to a valve wherein the valve comprises a Mitral valve or a tricuspid valve.

7. The valve clip removal system of claim 5, wherein a combination of the first and second types of snares provides stability for a concentric orientation for the cutting sheath to progress over the valve clip.

8. The valve clip removal system of claim 5, further comprising:
wherein the second snare is a forceps-type snare that is extended to a top of the second section of the valve clip to secure the valve clip via a grasping action.

9. The valve clip removal system of claim 8, further comprising:
in response to the torque action caused to valve clip when the first section is secured by the lasso-type snare, the forceps-type snare is configured to attach to the first section of the valve clip and to exert the straight upward force to compensate for the offset direction caused by the torque action to the first section of the valve clip, and to redirect the valve clip for positioning so the cutting sheath can progress forward over the valve clip for the clip removal procedure.

10. The valve clip removal system of claim 9, further comprising:
the forceps-type snare is configured to provide a rail that enables the cutting sheath to slide over the forceps-type snare and to progress forward over the valve clip.

11. The valve clip removal system of claim 10, further comprising:
wherein in a venous-transseptal approach to access a left atrium for a valve clip removal because of a deflectability required by the cutting sheath for positioning concentrically over a valve clip, the lasso-type snare and the forceps-type snare are used.

12. The valve clip removal system of claim 11, further comprising:
wherein the cutting sheath is configured as a laser fiber bundle for a laser sheath or a laser-cut hypotube for a mechanical cutting sheath that contains the cutting mechanism for deflection and is insertable through a deflectable sheath of adequate size while achieving the deflectability required by the cutting sheath to access a valve for the valve clip removal.

13. A valve clip removal apparatus, comprising:
a catheter configured with a first lumen, and a second lumen;
a cutting sheath configured with a cutting mechanism;
wherein the first lumen is used to deploy a first snare that enables securing a lower part of a valve clip;
wherein the second lumen is used to deploy a second snare that enables securing of an upper part of the valve clip;
upon securing the first section of the valve clip, the second snare is configured to pull the cutting sheath over the upper part of the valve clip to enable the cutting sheath to hold the valve clip in a stable position and to detach the valve clip from a valve leaflet whilst the valve clip is held; and
after the valve clip is freed from the valve leaflet, the cutting sheath is configured for removal whilst the valve clip is held.

14. The valve clip removal apparatus of claim 13, further comprising:
wherein in a transapical approach to access the mitral valve for a valve clip removal, the first snare is configured as a lasso-type snare and the second snare is configured as a hook-type snare; and
wherein in a venous-transseptal approach to access a valve for the valve clip removal, the first snare is configured as the lasso-type snare and the second snare is configured as either a lasso type snare or a forceps-type snare.

15. The valve clip removal apparatus of claim 13, further comprising:
wherein in a venous-transseptal approach to access a valve for a valve clip removal, in a first instance, the first snare is configured as a lasso-type snare and the second snare is configured as a lasso snare, and in a second instance, the first snare is configured as a lasso snare and the second snare is configured as a forceps-type snare.

16. The valve clip removal apparatus of claim 15, further comprising:
in response to a torque action caused to valve clip when the lower part is secured by the lasso-type snare, either a hook-type snare or the forceps-type snare is configured to attach to the upper part of the valve clip and to pull or grasp the valve clip for a concentric orientation for a clip removal procedure.

17. A valve clip removal system comprising a pair of tool lumens snares composed of a first snare of a lasso-type snare, and a second snare that is either a hook-type snare or a forceps-type snare, and a cutting sheath to remove a valve clip, the valve clip removal system comprising:
wherein the cutting sheath is configured large enough to pass through at least a single snare with a cutting mechanism;
wherein the cutting sheath is used to pass through the first snare to secure a lower part of a valve clip;
wherein the cutting sheath is used to pass through the second snare to secure an upper part of the valve clip;
in response to securing the lower part of the valve clip, the second snare attaches to the upper part of the valve clip to pull the cutting sheath downward over the upper part of the valve clip for the cutting sheath to both hold and remove the valve clip from a valve leaflet whilst the valve clip is held; and
after the valve clip is detached from the valve leaflet, the cutting sheath is configured for removal whilst holding the valve clip.

18. The valve clip removal system of claim 17, further comprising:
wherein the second snare is not deployed and the valve clip is secured by only the first snare and the cutting sheath for a valve clip removal procedure.

19. The valve clip removal system of claim 17, further comprising:
in response to a torque action caused to valve clip when the lower part is secured by the lasso-type snare, either the hook-type snare or the forceps-type snare is configured to attach to the upper part of the valve clip and to pull or grasp the valve clip for a concentric orientation for a valve clip removal procedure.

20. The valve clip removal system of claim 18, further comprising:
wherein in a transapical approach to access the mitral valve for the valve clip removal procedure, the second snare is the hook-type snare; and
wherein in a venous-transseptal approach to access the mitral valve for the valve clip removal, the second snare is either a lasso or forceps-type snare.
